(19) United States
(12) Patent Application Publication
(10) Pub. No.: US 2025/0027948 A1
(43) Pub. Date: Jan. 23, 2025

(54) EXTRACTION METHOD AND USE OF CUCUMBER RIBOSOME

(71) Applicants: Shenzhen Second People's Hospital (Shenzhen Institute of Translational Medicine), Shenzhen (CN); Shenzhen Institute of Advanced Technology, Chinese Academy of Sciences, Shenzhen (CN)

(72) Inventors: Li DUAN, Shenzhen (CN); Bin ZENG, Shenzhen (CN); Hang ZHANG, Shenzhen (CN)

(73) Assignees: Shenzhen Second People's Hospital (Shenzhen Institute of Translational Medicine), Shenzhen (CN); Shenzhen Institute of Advanced Technology, Chinese Academy of Sciences, Shenzhen (CN)

(21) Appl. No.: 18/515,287

(22) Filed: Nov. 21, 2023

(30) Foreign Application Priority Data
Jul. 17, 2023 (CN) 202310874177.3

(51) Int. Cl. G01N 33/68

(52) U.S. Cl. CPC G01N 33/68; G01N 33/6848

(57) ABSTRACT

A ribosome extraction method is provided. In the ribosome extraction method, cucumber cubes and a phosphate buffer saline (PBS) solution are mixed and slurried, and an 80S cucumber ribosome is prepared by first centrifugation, second centrifugation, third centrifugation, ultracentrifugation, and fourth centrifugation. The extracted 80S cucumber ribosome is relatively complete and high in yield, and about 2400 OD260 of 80S ribosome can be extracted from 500 g of cucumbers, so that the problems of difficult extraction and low yield of the ribosome are solved, and a foundation is laid for protein structure determination and function analysis.

### CROSS REFERENCE TO THE RELATED APPLICATIONS

This application is based upon and claims priority to Chinese Patent Application No. 202310874177.3, filed on Jul. 17, 2023, the entire contents of which are incorporated herein by reference.

### TECHNICAL FIELD

The present invention relates to the technical field of ribosome extraction, and in particular, to an extraction method and use of a cucumber ribosome.

### BACKGROUND

Ribosomes are intracellular ribonucleoprotein particles that are mainly composed of RNA and proteins. Ribosomes play an important role in processes such as mRNA translation, protein folding, peptidyl transfer, and peptidyl hydrolysis. Analysis of the structure and mechanism of action of ribosomes can help clarify the mechanisms of protein synthesis and modification, and thus, understanding the working mechanism of ribosomes is of great significance to understanding life. Professor Thomas A. Steitz also won the Nobel Prize in Chemistry for his contributions to the analysis of ribosome structure and function. The preparation of high-purity and biologically active protein samples is the basis and precondition for protein structure analysis and functional analysis.

The widely used method for extracting polyribosomes at present mainly comprises sucrose density gradient centrifugation and immunoprecipitation. Sucrose density gradient centrifugation is mainly implemented by adding homogenate to a sucrose density gradient solution after cells are crushed and filtered, and performing centrifugation using an ultracentrifuge. Although this method has a high yield, this method has high requirements on equipment and is complex to operate. The use of vertical rotor density gradient ultracentrifugation has been reported in the literature for rapid fractionation of polyribosomes, however, the requirements on equipment are also high. The existing immunoprecipitation has been applied in the plant Arabidopsis thaliana; however, the yield of ribosomes in this method is low, and therefore, this method is not suitable for subsequent in-depth research.

### SUMMARY

An objective of the present invention is to provide an extraction method of a cucumber ribosome. This extraction method is simple and convenient to operate and can efficiently extract a large amount of cucumber ribosomes.

In order to achieve the above objective, the present invention provides the following technical solutions.

The present invention provides an extraction method of a cucumber ribosome, comprising the following steps:

S1: mixing cucumber cubes with a phosphate buffer saline (PBS) solution, and squeezing to obtain a mixture; wherein a weight ratio of the cucumber cubes to the PBS solution is 4-7:25;

S2: performing first centrifugation on the mixture obtained in the step S1, and taking a supernatant 1; wherein the first centrifugation is performed at a centrifugal force of 2000-3000 g for 20-30 min;

S3: performing second centrifugation on the supernatant 1 obtained in the step S2, and taking a supernatant 2; wherein the second centrifugation is performed at a centrifugal force of 5000-6000 g for 60-90 min;

S4: performing third centrifugation on the supernatant 2 obtained in the step S3, and taking a supernatant 3; wherein the third centrifugation is performed at a centrifugal force of 9000-10000 g for 60-90 min;

S5: filtering the supernatant 3 obtained in the step S4 by using a filter membrane to obtain a filtrate;

S6: ultracentrifuging the filtrate obtained in the step S5, and discarding a supernatant to obtain a precipitate; wherein the ultracentrifugation is performed at a centrifugal force of 100000-140000 g for 60-90 min; and

S7: mixing the precipitate obtained in the step S6 with a PBS solution, performing fourth centrifugation after resuspension, and taking a supernatant to obtain a cucumber ribosome suspension; wherein the fourth centrifugation is performed at a centrifugal force of 14000-16000 g for 15-30 min.

Preferably, the PBS solutions in the steps S1 and S7 have a concentration of 0.01-0.02 M.

Preferably, the first centrifugation, the second centrifugation, the third centrifugation, the ultracentrifugation, and the fourth centrifugation are performed at a temperature of 3-5° C.

Preferably, the squeezing is performed for 1-5 min.

Preferably, a ratio of the PBS solution in the step S7 to the precipitate in the step S6 is 1-5 μL: 1 μg.

Preferably, the filter membrane in the step S5 has a pore size of 0.21-0.23 μm.

The present invention further provides a cucumber ribosome obtained by the extraction method.

The present invention further provides use of the cucumber ribosome in protein structure analysis and functional analysis.

By using the above technical solutions, the present invention has the following beneficial effects.

The present invention simply and efficiently prepares a large amount of cucumber ribosomes by combining differential centrifugation and ultracentrifugation, and the prepared cucumber ribosomes can be used in protein structure analysis and functional analysis.

### BRIEF DESCRIPTION OF THE DRAWINGS

FIG. 1 is a cryo-electron micrograph of a cucumber ribosome.

FIG. 2 is a cryo-electron microscopy density map of a cucumber ribosome.

FIG. 3 shows a local resolution of a cryo-electron microscopy density map of a cucumber ribosome (different colors represent different resolutions).

### DETAILED DESCRIPTION OF THE EMBODIMENTS

The present invention provides an extraction method of a cucumber ribosome, comprising the following steps:

S1: mixing cucumber cubes with a PBS solution, and squeezing to obtain a mixture; wherein a weight ratio of the cucumber cubes to the PBS solution is 4-7:25;

S2: performing first centrifugation on the mixture obtained in the step S1, and taking a supernatant 1; wherein the first centrifugation is performed at a centrifugal force of 2000-3000 g for 20-30 min;

S3: performing second centrifugation on the supernatant 1 obtained in the step S2, and taking a supernatant 2; wherein the second centrifugation is performed at a centrifugal force of 5000-6000 g for 60-90 min;

S4: performing third centrifugation on the supernatant 2 obtained in the step S3, and taking a supernatant 3; wherein the third centrifugation is performed at a centrifugal force of 9000-10000 g for 60-90 min;

S5: filtering the supernatant obtained in the step S4 by using a filter membrane to obtain a filtrate;

S6: ultracentrifuging the filtrate obtained in the step S5, and discarding a supernatant to obtain a precipitate; wherein the ultracentrifugation is performed at a centrifugal force of 100000-140000 g for 60-90 min; and

S7: mixing the precipitate obtained in the step S6 with a PBS solution, performing fourth centrifugation after resuspension, and taking a supernatant to obtain a cucumber ribosome suspension; wherein the fourth centrifugation is performed at a centrifugal force of 14000-16000 g for 15-30 min.

In the present invention, cucumber is peeled, washed by water and cut into cubes to obtain cucumber cubes; and the water is preferably DEPC treated double distilled water.

In the present invention, cucumber cubes are mixed with a PBS solution, and squeezed to obtain a mixture; a weight ratio of the cucumber cubes to the PBS solution is preferably 4-7:25, and further preferably 5-6:25; the PBS solution has a concentration of preferably 0.01-0.02 M, and further preferably 0.015 M; the squeezing is performed for preferably 2-5 min, further preferably 3-4 min, and more preferably 3.5 min; and the squeezing is preferably performed in a juicer. In the present invention, the operation temperature after the squeezing is preferably 3-5° C., and more preferably 4° C.

In the present invention, the first centrifugation is performed on the mixture obtained in the step S1, and a supernatant 1 is taken; the first centrifugation has a centrifugal force of preferably 2000-3000 g, further preferably 2200-2800 g, and more preferably 2500 g; the first centrifugation is performed for preferably 20-30 min, further preferably 23-28 min, and more preferably 25 min; and the purpose of the first centrifugation is to remove larger plant fibers.

In the present invention, the second centrifugation is performed on the supernatant 1, and a supernatant 2 is taken; the second centrifugation has a centrifugal force of preferably 5000-6000 g, further preferably 5300-5700 g, and more preferably 5500 g; the second centrifugation is performed for preferably 60-90 min, further preferably 70-80 min, and more preferably 75 min; and the purpose of the second centrifugation is to remove cell debris and cell nuclei.

In the present invention, the third centrifugation is performed on the supernatant 2, and a supernatant 3 is taken; the third centrifugation has a centrifugal force of preferably 9000-10000 g, further preferably 9400-9800 g, and more preferably 9600 g; the third centrifugation is performed for preferably 60-90 min, further preferably 65-82 min, and more preferably 70 min, and the purpose of the third centrifugation is to remove mitochondria.

In the present invention, the supernatant 3 is filtered by a filter membrane, and the filtrate is taken; and the filter membrane has a pore size of 0.21-0.23 μm, and more preferably 0.22 μm.

In the present invention, the ultracentrifugation is performed on the filtrate, and a supernatant is discarded to obtain a precipitate; the ultracentrifugation has a centrifugal force of preferably 100000-140000 g, further preferably 110000-130000 g, and more preferably 120000 g; the ultracentrifugation is performed for preferably 60-90 min, further preferably 67-85 min, and more preferably 77 min; and the purpose of ultracentrifugation is to further remove smaller plant fibers and cell debris.

In the present invention, the precipitate is mixed with a PBS solution and then is resuspended to obtain cucumber ribosome crude extract; a mixing ratio of the PBS solution to the precipitate is preferably 1-5 μL: 1 μg, further preferably 2-4 μL: 1 μg, and more preferably 3 μL: 1 μg; and the PBS solution has a concentration of preferably 0.01-0.02 M, and more preferably 0.015 M.

In the present invention, the fourth centrifugation is performed on the cucumber ribosome crude extract, and a supernatant 4 is taken, namely, 80S cucumber ribosome; the fourth centrifugation has a centrifugal force of preferably 14000-16000 g, further preferably 14500-15500 g, and more preferably 15000 g; and the fourth centrifugation is performed for preferably 15-30 min, further preferably 20-25 min, and more preferably 23 min.

The present invention further provides a cucumber ribosome obtained by the extraction method.

The present invention further provides use of the cucumber ribosome described above in protein structure analysis and functional analysis.

The technical solutions provided by the present invention will be described in detail below with reference to the examples, which, however, should not be construed as limiting the scope of the present invention.

### Example 1

500 g of cucumbers were peeled, washed with DEPC treated double distilled water 3 times and cut into cucumber cubes; and the cucumber cubes were mixed with 1.8 L PBS solution with a concentration of 0.01 M, and squeezed in a juicer for 2 min to obtain homogenate. The homogenate was centrifuged at 2000 g for 20 min to remove the larger plant fibers; the supernatant was removed, and the homogenate was centrifuged at 5000 g for 60 min to remove cell debris and cell nuclei; and the supernatant was removed again, and then the homogenate was centrifuged at 9000 g for 60 min to remove mitochondria. Finally, the supernatant was filtered through a 0.22 μm filter membrane and centrifuged at 100000 g for 60 min, the supernatant was discarded, 180 μL 0.01 M of the PBS solution was added, and the mixture was resuspended to obtain the cucumber ribosome crude extract. The crude cucumber ribosome crude extract was centrifuged at 14000 g for 15 min, and the supernatant was 80S cucumber ribosomes.

### Example 2

500 g of cucumbers were peeled, washed with DEPC treated double distilled water 2 times and cut into cucumber cubes; and the cucumber cubes were mixed with 2.5 L PBS solution with a concentration of 0.01 M, and squeezed in a juicer for 3 min to obtain homogenate. The homogenate was centrifuged at 2200 g for 26 min to remove the larger plant fibers; the supernatant was removed, and the homogenate was centrifuged at 5600 g for 70 min to remove cell debris and cell nuclei; and the supernatant was removed again, and then the homogenate was centrifuged at 9500 g for 80 min to remove mitochondria. Finally, the supernatant was filtered through a 0.22 μm filter membrane and centrifuged at 120000 g for 75 min, the supernatant was discarded, 200 μL 0.015 M of the PBS solution was added, and the mixture was resuspended to obtain the cucumber ribosome crude extract. The crude cucumber ribosome crude extract was centrifuged at 15000 g for 20 min, and the supernatant was 80S cucumber ribosomes.

### Example 3

500 g of cucumbers were peeled, washed with DEPC treated double distilled water 3 times and cut into cucumber cubes; and the cucumber cubes were mixed with 3.1 L PBS solution with a concentration of 0.02 M, and squeezed in a juicer for 5 min to obtain homogenate. The homogenate was centrifuged at 3000 g for 30 min to remove the larger plant fibers; the supernatant was removed, and the homogenate was centrifuged at 6000 g for 90 min to remove cell debris and cell nuclei; and the supernatant was removed again, and then the homogenate was centrifuged at 10000 g for 90 min to remove mitochondria. Finally, the supernatant was filtered through a 0.23 μm filter membrane and centrifuged at 140000 g for 90 min, the supernatant was discarded, 220 μL 0.02 M of the PBS solution was added, and the mixture was resuspended to obtain the cucumber ribosome crude extract. The crude cucumber ribosome crude extract was centrifuged at 16000 g for 30 min, and the supernatant was 80S cucumber ribosomes.

### Comparative Example 1

This comparative example is different from Example 1 in that the centrifugal force of the first centrifugation is 1500 g.

### Comparative Example 2

This comparative example is different from Example 1 in that the centrifugal force of the first centrifugation is 4000 g.

### Comparative Example 3

This comparative example is different from Example 1 in that the centrifugal force of the second centrifugation is 3500 g.

### Comparative Example 4

This comparative example is different from Example 1 in that the centrifugal force of the second centrifugation is 7000 g.

### Comparative Example 5

This comparative example is different from Example 1 in that the centrifugal force of the third centrifugation is 8000 g.

### Comparative Example 6

This comparative example is different from Example 1 in that the centrifugal force of the third centrifugation is 11000 g.

### Comparative Example 7

This comparative example is different from Example 1 in that the centrifugal force of the ultracentrifugation is 90000 g.

### Comparative Example 8

This comparative example is different from Example 1 in that the centrifugal force of the ultracentrifugation is 150000 g.

### Comparative Example 9

This comparative example is different from Example 1 in that the centrifugal force of the fourth centrifugation is 12000 g.

### Comparative Example 10

This comparative example is different from Example 1 in that the centrifugal force of the fourth centrifugation is 18000 g.

The cucumber 80S ribosome finally extracted by the present invention is viscous liquid and slightly green, and the measurement result of the ultraviolet spectrophotometer measurement of the cucumber ribosome prepared in Example 1 is as follows: OD260/OD280=1.80, which indicates that the extracted cucumber 80S ribosomes are relatively intact, because a clear ribosomal solution will have an absorbance ratio: OD260/OD280=1.80-1.85. About 2400 OD260 of 80S ribosome can be prepared from 500 g of cucumbers, so that an amount of cucumber 80S ribosomes extracted using this method is larger.

In Comparative Examples 1 to 10, cucumber ribosomes cannot be finally separated because the centrifugal force at a certain step is not within the scope of the present invention.

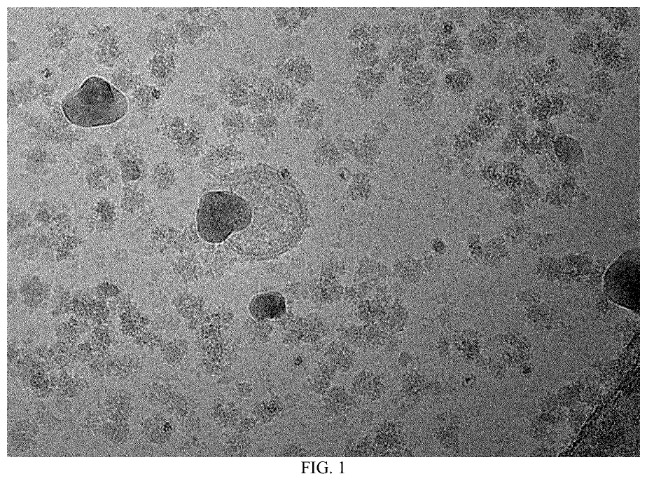

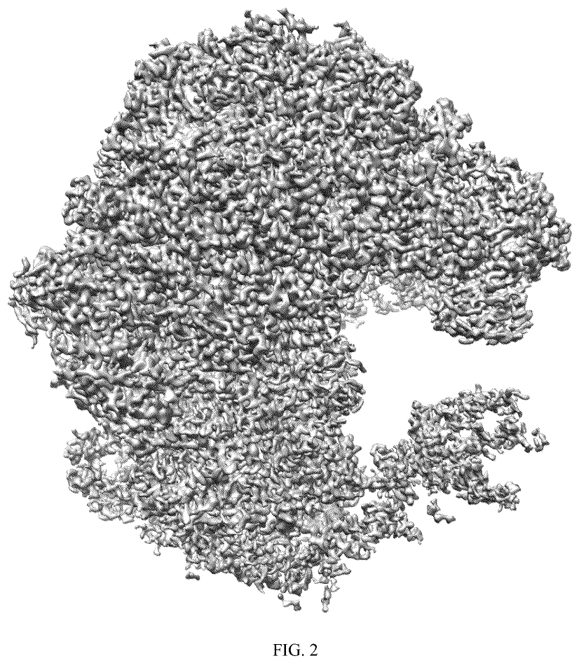

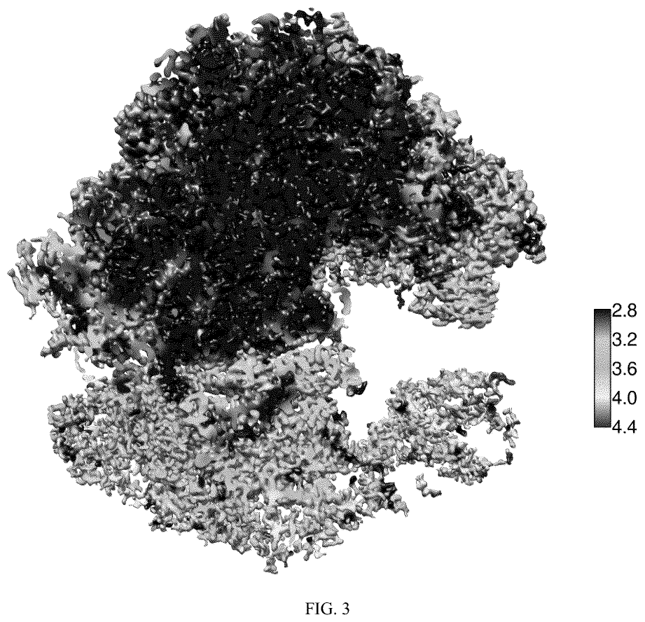

The shape of the cucumber ribosome extracted in Example 1 is identified by cryo-electron microscopy, as shown in FIG. 1; the cryoSPARC is used for processing the data of the cryo-electron microscopy, the rough flow is as follows: protein particles picking and extraction, two-dimensional classification and selection, initial model establishment, three-dimensional classification and three-dimensional model refinements, and finally a cryo-EM density map containing intact cucumber ribosomes with large and small subunits is obtained. The cryo-EM density map of cucumber ribosomes is shown in FIG. 2, and the local resolution of the density map is shown in FIG. 3.

The above descriptions are only preferred embodiments of the present invention. It should be noted that those of ordinary skill in the art can also make several improvements and modifications without departing from the principle of the present invention, and such improvements and modifications shall fall within the protection scope of the present invention.

What is claimed is:

1. An extraction method of a cucumber ribosome, comprising the following steps:
S1: mixing cucumber cubes with a first phosphate buffer saline (PBS) solution, and squeezing to obtain a mixture; wherein a weight ratio of the cucumber cubes to the first PBS solution is 4-7:25;
S2: performing a first centrifugation on the mixture obtained in S1, and taking a first supernatant; wherein the first centrifugation is performed at a centrifugal force of 2000-3000 g for 20-30 min;
S3: performing a second centrifugation on the first supernatant obtained in S2, and taking a second supernatant; wherein the second centrifugation is performed at a centrifugal force of 5000-6000 g for 60-90 min;
S4: performing a third centrifugation on the second supernatant obtained in S3, and taking a third supernatant; wherein the third centrifugation is performed at a centrifugal force of 9000-10000 g for 60-90 min;
S5: filtering the third supernatant obtained in S4 by using a filter membrane to obtain a filtrate;
S6: performing an ultracentrifugation on the filtrate obtained in S5, and discarding a fourth supernatant to obtain a precipitate; wherein the ultracentrifugation is performed at a centrifugal force of 100000-140000 g for 60-90 min; and
S7: mixing the precipitate obtained in S6 with a second PBS solution, performing a fourth centrifugation after a resuspension, and taking a fifth supernatant to obtain a cucumber ribosome suspension; wherein the fourth centrifugation is performed at a centrifugal force of 14000-16000 g for 15-30 min.

2. The extraction method according to claim 1, wherein the first PBS solution in S1 and the second PBS solution in S7 have a concentration of 0.01-0.02 M.

3. The extraction method according to claim 2, wherein the first centrifugation, the second centrifugation, the third centrifugation, the ultracentrifugation, and the fourth centrifugation are performed at a temperature of 3-5° C.

4. The extraction method according to claim 1, wherein the squeezing is performed for 1-5 min.

5. The extraction method according to claim 4, wherein a ratio of the second PBS solution in S7 to the precipitate obtained in S6 is 1-5 μL: 1 μg.

6. The extraction method according to claim 5, wherein the filter membrane in S5 has a pore size of 0.21-0.23 μm.

7. A cucumber ribosome obtained by the extraction method according to claim 1.

8. A method of using the cucumber ribosome according to claim 7 in protein structure determination and functional analysis.

9. The cucumber ribosome according to claim 7, wherein in the extraction method, the first PBS solution in S1 and the second PBS solution in S7 have a concentration of 0.01-0.02 M.

10. The cucumber ribosome according to claim 9, wherein in the extraction method, the first centrifugation, the second centrifugation, the third centrifugation, the ultracentrifugation, and the fourth centrifugation are performed at a temperature of 3-5° C.

11. The cucumber ribosome according to claim 7, wherein in the extraction method, the squeezing is performed for 1-5 min.

12. The cucumber ribosome according to claim 11, wherein in the extraction method, a ratio of the second PBS solution in S7 to the precipitate obtained in S6 is 1-5 μL: 1 μg.

13. The cucumber ribosome according to claim 12, wherein in the extraction method, the filter membrane in S5 has a pore size of 0.21-0.23 μm.
